(19) United States
(12) Patent Application Publication
(10) Pub. No.: US 2026/0274840 A1
(43) Pub. Date: Sep. 17, 2026

(54) POLYMORPHS OF AN INTEGRIN Alpha5Beta1

(71) Applicant: ELI LILLY AND COMPANY, Indianapolis, IN (US)

(72) Inventors: David Andrew COATES, New Palestine, IN (US); Gislaine KUMINEK, Brownsburg, IN (US)

(21) Appl. No.: 19/560,754

(22) Filed: Mar. 9, 2026

Related U.S. Application Data
(60) Provisional application No. 63/770,608, filed on Mar. 12, 2025.
(60) Provisional application No. 63/979,223, filed on Feb. 10, 2026.

(51) Int. Cl. C07D 471/04; A61K 31/4375

(52) U.S. Cl. CPC C07D 471/04; A61K 31/4375

(57) ABSTRACT

Provided herein are (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorphs, salts, compositions, methods of preparation, and use thereof.

### FIELD OF THE INVENTION

Provided herein are polymorphs and salts of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid compositions thereof, methods of preparation thereof, and methods of use thereof.

### BACKGROUND OF THE INVENTION

Fibronectin (Fn) is an extracellular matrix protein that orchestrates complex cell adhesion and signaling through cell surface integrin receptors (Fibronectin-binding integrins (e.g., α5β1)) during tissue development, remodeling, and disease, such as hypertension and heart failure. Integrins are a family of glycoprotein transmembrane receptors that mediate cell-cell and cell-matrix interactions.

Integrin α5β1 is composed of subunits ITGA5 (integrin α5) and integrin β1. Several integrins bind to fibronectin. Integrin α5β1 is selective for fibronectin since it requires both the 9^{th} and 10^{th} type II repeats of fibronectin (FNIII-9 and FNIII-10) for interaction. Expression of α5β1 integrin is mainly in the vasculature and connective tissue. The integrin α5β1 (a5b1 or alpha5 beta1) is composed of an α5 (a5 or alpha5) and β1 (b1 or beta1) subunit. The a5 subunit forms a specific dimer with the beta1 subunit, and is widely expressed in most tissues. Integrin α5β1 almost exclusively mediates cell adhesion through an interaction with fibronectin, binding via the short arginine-glycine-aspartate (RGD) adhesion motif. Endothelial cells and platelets can however bind to fibrin via α5β1. The α5β1 interaction with fibronectin plays an important role in physiopathological angiogenesis and vascular integrity. The compound (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid (Compound 1), is described by preparations herein. Compound 1 can inhibit integrin α5β1. The structure of Compound 1 is provided:

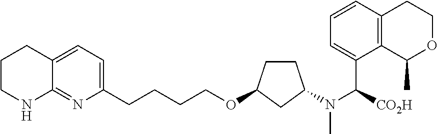 [SMILES: C[C@@H]1OCCc2cccc([C@@H](C(=O)O)N(C)[C@H]3CC[C@H](OCCCCc4ccc5c(n4)NCCC5)C3)c21]

### Compound 1.

A polymorph of Compound 1 or a salt thereof may provide the advantages of bioavailability and stability and may be suitable for use as an active agent in a pharmaceutical composition. There is a need for a polymorph of Compound 1 with desired relative stability and control of form interconversion to provide a suitable polymorph for large scale production, transportation, storage, and pre-usage preparation. The ability to control and produce a relatively stable polymorph with robust manufacturing processes is generally required for regulatory approval and marketing; however, crystalline solid forms can have unanticipated and surprising physical properties. There is a need for a polymorph and/or salt form of Compound 1 enabling desired bioavailability and manufacturability. Alternative solid forms may be necessary for large-scale production processes. Accordingly, there is a need for various new salt or polymorphic forms of Compound 1 with desired chemical and physical stabilities, and compositions and uses of the same. Therefore, a polymorphic form of Compound 1 that is suitable for large scale manufacture is desired. A polymorphic form of Compound 1 providing a higher melting point and/or greater stability is also desired. There is a desire for a salt form suitable for manufacture and acceptable shelf-life stability.

### SUMMARY OF THE INVENTION

In one aspect, provided herein is a(S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, polymorph or a pharmaceutically acceptable salt thereof.

In an aspect, provided herein is a (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, polymorph or a pharmaceutically acceptable salt thereof, with water present.

In one aspect, provided herein is a polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized as having an X-ray powder diffraction pattern using Cu Kα radiation comprising a peak of 4.6±0.2 2θ and at least one peak selected from the group consisting of, 11.9, and 17.2±0.2 2θ.

In one aspect, provided herein is a polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized as having an X-ray powder diffraction pattern using Cu Kα radiation comprising peaks of 4.6, 11.9, and 17.2±0.2 2θ.

The polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized as having an x-ray powder diffraction pattern using Cu Kα radiation comprising peaks of 4.6, 11.2, 11.9, 14.1, 14.6, 17.2, and 22.7±0.2 2θ.

The polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized as having an XRPD pattern substantially as shown in FIG. 1.

The polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized by a solid state ¹³C NMR spectrum comprising a peak at 21.3 ppm, ±0.4 ppm.

The polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized by a solid state ¹³C NMR spectrum comprising a peak at 21.3 ppm, ±0.4 ppm, and at least one selected from the group consisting of 67.5 and 179.9 ppm±0.4 ppm.

The polymorph of_(S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized by a solid state ¹³C NMR spectrum comprising peaks at 21.3, 67.5, and 179.9 ppm, ±0.4 ppm.

The polymorph of_(S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized as having a ¹³C NMR spectrum substantially as shown in FIG. 7.

The polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized as having an XRPD pattern substantially as shown in FIG. 2.

The polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized by a solid state ¹³C NMR spectrum comprising a peak at 26.2 ppm, ±0.4 ppm.

The polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized by a solid state ¹³C NMR spectrum comprising a peak at 26.2 ppm, ±0.4 ppm, and at least one selected from the group consisting of 70.1 and 171.1 ppm±0.4 ppm.

The polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized by a solid state ¹³C NMR spectrum comprising peaks at 26.2, 70.1, and 171.1 ppm, ±0.4 ppm.

The polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized as having a ¹³C NMR spectrum substantially as shown in FIG. 6.

The polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized by a solid state ¹³C NMR spectrum comprising a peak at 153.7 ppm, ±0.4 ppm.

The polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized by a solid state ¹³C NMR spectrum comprising a peak at 153.7 ppm, ±0.4 ppm, and at least one selected from the group consisting of 31.0 and 58.0 ppm±0.4 ppm.

The polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized by a solid state ¹³C NMR spectrum comprising peaks at 31.0, 58.0, and 153.7 ppm, ±0.4 ppm.

The polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized as having a ¹³C NMR spectrum substantially as shown in FIG. 8.

A method for preparing a polymorph (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, comprising converting the (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid naproxen salt to a polymorph.

A compound comprising (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid naproxen salt.

A pharmaceutical composition comprising a polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, and a pharmaceutically acceptable carrier.

The pharmaceutical composition comprising (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form M, wherein the pharmaceutical composition is substantially free of other polymorphic forms of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid or a pharmaceutically acceptable salt thereof.

A method of treating hypertension in a subject in need thereof, comprising administering an effective amount of the polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid.

A method of treating hypertension in a subject in need thereof, comprising administering an effective amount of a composition comprising a polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid and a pharmaceutically acceptable carrier.

A method of treating pulmonary arterial hypertension (PAH) in a subject in need thereof, comprising administering an effective amount of the polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid.

A method for treating pulmonary arterial hypertension (PAH) in a subject in need thereof, comprising administering an effective amount of a composition comprising a polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid and a pharmaceutically acceptable carrier.

In another aspect, provided herein are methods of preparing a polymorph of Compound 1.

In another aspect, provided herein are compositions comprising a polymorph of Compound 1.

In another aspect, provided herein are methods of treating pulmonary hypertension in a subject in need thereof, comprising administering an effective amount of a polymorph of Compound 1.

In another aspect, provided herein are methods of treating pulmonary arterial hypertension (PAH) in a subject in need thereof, comprising administering an effective amount of a polymorph of Compound 1.

In one aspect, provided herein is a (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph characterized as having an x-ray powder diffraction pattern using Cu Ku radiation comprising a peak at 4.6, and at least one peak selected from the group consisting of 11.9, and 17.2±0.2 2θ.

In one aspect provided herein is a compound characterized as having an x-ray powder diffraction pattern using Cu Kα radiation comprising at least the peaks at 4.6, 11.9, and 17.2±0.2 2θ.

In one aspect provided herein is a polymorph Form G of Compound 1, characterized as having an x-ray powder diffraction pattern using Cu Kα radiation comprising at least the peaks at 4.6, 11.9, and 17.2±0.2 2θ.

In another aspect, provided herein are methods of preparing a polymorph Form G.

In another aspect, provided herein are compositions comprising a polymorph Form G.

In another aspect, provided herein are methods of treating pulmonary hypertension in a subject in need thereof, comprising administering an effective amount of a polymorph Form G.

In another aspect, provided herein are methods of treating pulmonary arterial hypertension (PAH) in a subject in need thereof, comprising administering an effective amount of a polymorph Form G.

In one aspect, provided herein is a polymorph Compound 1, characterized as having an x-ray powder diffraction pattern using Cu Kα radiation comprising a peak of 7.7±0.2 and at least one peak selected from the group consisting of 9.7 and 14.8±0.2 2θ.

In one aspect, provided herein is a polymorph Form M of Compound 1, characterized as having an x-ray powder diffraction pattern using Cu Kα radiation comprising at least the peaks of 7.7, 9.7, and 14.8±0.2 2θ.

In another aspect, provided herein is a polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, characterized as having an x-ray powder diffraction pattern using Cu Kα radiation comprising at least peaks of 7.7, 9.7, 13.5, 14.8, and 16.8±0.2 2θ.

In another aspect, provided herein are methods of preparing a polymorph Form M.

In another aspect, provided herein are compositions comprising a polymorph Form M.

In another aspect, provided herein are methods of treating pulmonary hypertension in a subject in need thereof, comprising administering an effective amount of a polymorph Form M.

In another aspect, provided herein are methods of treating pulmonary arterial hypertension (PAH) in a subject in need thereof, comprising administering an effective amount of a polymorph Form M.

In one aspect, provided herein is a (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid naproxen salt.

A naproxen salt may be useful for preparation of certain polymorphs of Compound 1. A process for preparation of polymorph Form G is desired. A process for preparation of polymorph Form M is desired.

In another aspect, provided herein is a methods for preparing a polymorph Form G comprising the preparation of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form G, wherein there is first prepared a solution of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid naproxen salt in a solvent or mixture of solvents and thereafter the (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form G is precipitated from the solution.

In another aspect, provided herein is a methods for preparing a polymorph Form M comprising the preparation of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form G, wherein there is first prepared a solution of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid naproxen salt in a solvent or mixture of solvents and thereafter the (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form M is precipitated from the solution.

In another aspect, provided herein are compositions comprising a polymorph Form G.

In another aspect, provided herein are methods of treating pulmonary hypertension in a subject in need thereof, comprising administering an effective amount of a polymorph Form G.

In another aspect, provided herein are methods of treating pulmonary arterial hypertension (PAH) in a subject in need thereof, comprising administering an effective amount of a polymorph Form G.

In another aspect, provided herein is a compound comprising (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid hippurate.

In another aspect, provided herein is a compound comprising a Hippurate (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid is characterized as having an XRPD pattern substantially as shown in FIG. 5.

### BRIEF DESCRIPTION OF THE DRAWINGS

FIG. 1 shows an X-ray powder diffraction (XRPD) pattern of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form G.

FIG. 2 shows an X-ray powder diffraction (XRPD) pattern of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form M.

FIG. 3 shows Differential Scanning Calorimetry (“DSC”) of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form G.

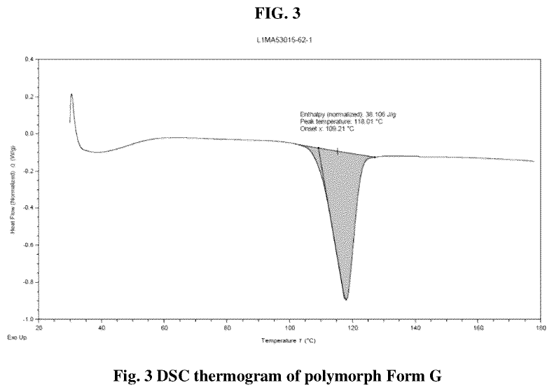

FIG. 4 shows Differential Scanning Calorimetry (“DSC”) of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form M.

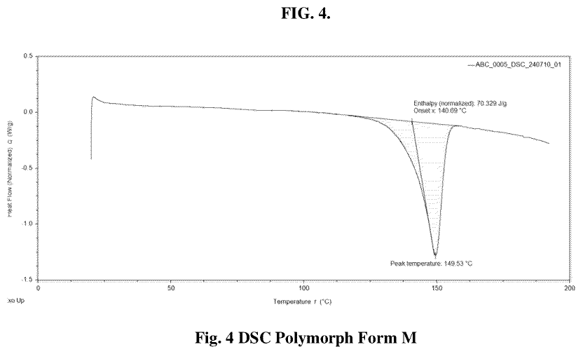

FIG. 5 shows an X-ray powder diffraction (XRPD) pattern of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid hippurate.

FIG. 6 shows C¹³ solid state NMR of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form M.

FIG. 7 shows C¹³ solid state NMR of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form G.

FIG. 8 shows C¹³ solid state NMR of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid amorphous.

### DETAILED DESCRIPTION OF THE INVENTION

As used herein and in the appended claims, the singular forms “a”, “an” and “the” include plural forms, unless the context clearly dictates otherwise.

As used herein, and unless otherwise specified, the terms “about” and “approximately,” when used in connection with doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in connection with a value, contemplate a variation within ±15%, within ±10%, within ±5%, within ±4%, within ±3%, within ±2%, within ±1%, or within ±0.5% of the specified value. Reference to “about” a value or parameter herein includes (and describes) embodiments that are directed to that value or parameter per se. For example, description referring to “about X” includes and describes embodiments that are directed to the value or parameter of “X per se”.

As used herein “XRPD” means x-ray powder diffraction using Cu Kα radiation.

As used herein, the term “polymorph” or “polymorphic form” refers to a crystalline form of a compound. Different polymorphs may have different physical properties such as, for example, melting temperatures, heats of fusion, solubilities, dissolution rates, and/or vibrational spectra as a result of the arrangement or conformation of the molecules or ions in the crystal lattice. The differences in physical properties exhibited by polymorphs may affect pharmaceutical parameters, such as storage stability, compressibility, density (important in formulation and product manufacturing), and dissolution rate (an important factor in bioavailability).

As used herein, the term “pharmaceutically acceptable carrier,” and cognates thereof, refers to adjuvants, binders, diluents, etc. known to the skilled artisan that are suitable for administration to an individual (e.g., a mammal or non-mammal). Combinations of two or more carriers are also contemplated. The pharmaceutically acceptable carrier(s) and any additional components, as described herein, should be compatible for use in the intended route of administration (e.g., oral or parenteral) for a particular dosage form, as would be recognized by the skilled artisan.

As used herein, “treatment” or “treating” is an approach for obtaining beneficial or desired results including clinical results. For purposes of this disclosure, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of this disclosure contemplate any one or more of these aspects of treatment.

As used herein, the term “substantially as shown in” when referring, for example, to an XRPD pattern, a DSC graph, or a TGA graph, includes a pattern or graph that is not necessarily identical to those depicted herein, but falls within the limits of experimental errors or deviations when considered by one of ordinary skill in the art.

As used herein, the term “substantially free of” means that the composition contains the indicated substance or substances in an amount of less than about 25%, less than about 20%, less than about 15%, less than about 10%, less than about 5%, less than about 4%, less than about 3%, less than about 2%, or less than about 1% by weight. In certain embodiments, “substantially free of” means that the composition contains the indicated substance or substances in an amount of less than about 10%, less than about 5%, less than about 4%, less than about 3%, less than about 2%, or less than about 1% by weight.

The term “subject” refers to an animal, including, but are not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

PCT application number PCT/US2024/047672 describes (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid (Compound 1).

As used herein, the term “effective amount” refers to an amount of a compound or composition sufficient to treat a specified disorder, condition or disease such as to ameliorate, to palliate, to lessen, and/or to delay one or more of its symptoms.

Variations in the crystal structure of a pharmaceutical drug substance may affect the dissolution rate (which may affect bioavailability, etc.), manufacturability (e.g., ease of handling, ease of purification, ability to consistently prepare doses of known strength, etc.) and stability (e.g., thermal stability, shelf life (including resistance to degradation), etc.) of a pharmaceutical drug product. Such variations may affect the methods of preparation or formulation of pharmaceutical compositions in different dosage or delivery forms, such as solid oral dosage forms including tablets and capsules. Compared to other forms such as non-crystalline or amorphous forms, polymorphs may provide desired or suitable hygroscopicity, particle size control, dissolution rate, solubility, purity, physical and chemical stability, manufacturability, yield, reproducibility, and/or process control. Thus, polymorphs of Compound 1 may provide advantages of improving the manufacturing process of an active agent or the stability or storability of a drug product form of the active agent, or having suitable bioavailability and/or stability as an active agent.

The use of certain conditions, such as the use of different solvents and/or temperatures, has been found to produce different polymorphs of Compound 1, including polymorphic Form G and Form M, described herein, which may exhibit one or more favorable characteristics described herein. In an embodiment is polymorphic Form M having greater chemical stability and/or higher melting point than other polymorphs of Compound 1. In an embodiment is polymorphic Form M having greater chemical stability and/or higher melting point than other polymorphs of Compound 1.

In certain embodiments, a salt form of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid may be desired. In an embodiment, (S)-2-(methyl((1S,3S)-3-(4-(5,6,7,8-tetrahydro-1,8-naphthyridin-2-yl)butoxy)cyclopentyl)amino)-2-((S)-1-methylisochroman-8 h-yl)acetic acid is a hippurate. In an embodiment, (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid is napthylacetic acid salt. In an embodiment, (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid is a naproxen salt.

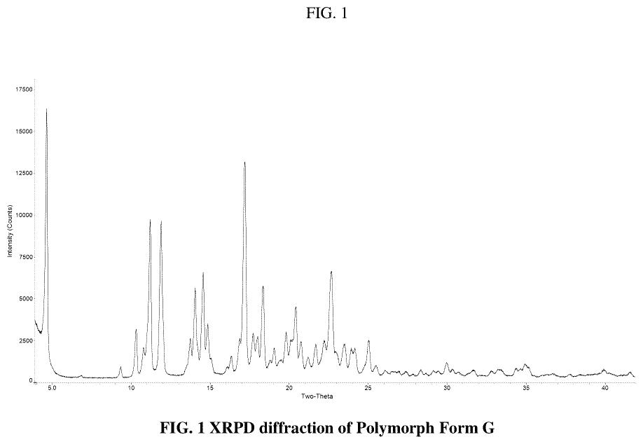

As used herein, “FaSSIF” means Fasted State Simulated Intestinal Fluid at pH=6.48. As used herein “FaSSGF” means Fasted State Simulated Gastric Fluid (FaSSGF) at pH=1.55. As used herein “FeSSIF” means Fed State Simulated Intestinal Fluid at pH=4.98.
In some embodiments (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form G has an XRPD pattern substantially as shown by FIG. 1. Angles 2-theta and relative peak intensities that may be observed for Form G using XRPD are shown in Table 1.

Table 1. X-ray powder diffraction peaks of the of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Form G

TABLE 1

(S)-2-(methyl((1S,3S)-3-(4-(5,6,7,8-tetrahydro-
1,8-naphthyridin-2-yl)butoxy)cyclopentyl)amino)-
2-((S)-1-methylisochroman-8-yl)acetic acid Form G

| | Angle (°2-Theta) | Relative Intensity |
|---|---|---|
| Peak | +/− 0.2° | (% of most intense peak) |
| 1 | 4.6 | 100.0% |
| 2 | 10.3 | 20.0% |
| 3 | 11.2 | 63.1% |
| 4 | 11.9 | 64.2% |
| 5 | 14.1 | 36.1% |
| 6 | 14.6 | 32.3% |
| 7 | 17.2 | 88.4% |
| 8 | 18.3 | 34.0% |
| 9 | 20.4 | 20.7% |
| 10 | 22.7 | 39.5% |

Angles 2-theta and relative peak intensities that may be observed for polymorphic Form G using XRPD are shown in Table 1. Angles 2-theta and relative peak intensities that may be observed for polymorphic Form M using XRPD are shown in Table 2.

Table 2. X-ray powder diffraction peaks of the (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Form M.

TABLE 2

(S)-2-(methyl((1S,3S)-3-(4-(5,6,7,8-tetrahydro-
1,8-naphthyridin-2-yl)butoxy)cyclopentyl)amino)-
2-((S)-1-methylisochroman-8-yl)acetic acid Form M

| | Angle (°2-Theta) | Relative Intensity |
|---|---|---|
| Peak | +/− 0.2° | (% of most intense peak) |
| 1 | 5.8 | 15.7% |
| 2 | 7.7 | 60.3% |
| 3 | 9.0 | 61.3% |
| 4 | 9.7 | 76.0% |
| 5 | 13.0 | 69.8% |
| 6 | 13.5 | 84.2% |
| 7 | 14.8 | 100.0% |
| 8 | 16.8 | 89.0% |
| 9 | 19.2 | 83.2% |
| 10 | 19.9 | 94.7% |

The XRPD patterns of crystalline solids are obtained on a Bruker D8 Endeavor X-ray powder diffractometer, equipped with a CuKα (1.5418 Å) source and a Linxeye detector, operating at 40 kV and 40 mA. The sample is scanned between 4 and 42 2θ°, with a step size of 0.009 2θ° and a scan rate of 0.5 seconds/step, and using 0.3° primary slit opening, and 3.9° PSD opening. The dry powder is packed on a quartz or silicon sample holder and a smooth surface is obtained using a glass slide. The crystal form diffraction patterns are collected at ambient temperature and relative humidity. Crystal peak positions are determined in MDI-Jade after whole pattern shifting based on an internal NIST 675 standard with peaks at 8.853 and 26.774 2θ°. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

Polymorph Form G and polymorph Form M were studied using Differential Scanning Calorimetry (“DSC”) performed using a TA Discovery DSC. The sample (1-2 mg) was weighed directly in a 40 μL hermetic aluminum pan with a pinhole and analyzed according to the parameters below:

Parameters

| Method | Ramp | |
|---|---|---|
| Sample size | 1-2 | mg |
| Heating rate | 10.0° | C./min |
| Temperature range | 30 to 250° | C. |
| Method gas | N₂ at 50.00 | mL/min |

In some embodiments, Form G has an XRPD pattern displaying at least two, at least three, at least four, at least five, at least six, at least seven, at least eight, at least nine, or at least ten of the peaks at angles 2-theta with the greatest intensity in the XRPD pattern as shown in FIG. 1, or as provided in Table 1.

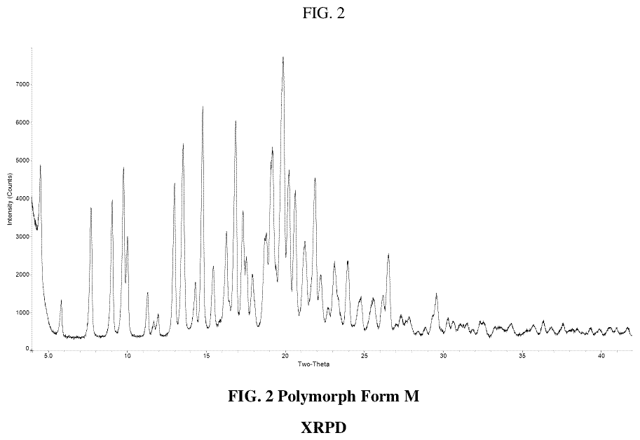

In some embodiments, Form M has an XRPD pattern displaying at least two, at least three, at least four, at least five, at least six, at least seven, at least eight, at least nine, or at least ten of the peaks at angles 2-theta with the greatest intensity in the XRPD pattern as shown in FIG. 2, or as provided in Table 2.

In some embodiments of the composition comprising polymorphic Form G, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total Compound 1 in the composition is polymorphic Form G.

In some embodiments of the composition comprising polymorphic Form M, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is polymorphic Form M.

In some embodiments of the composition comprising Form M, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound 1 exists in polymorphic Form M.

As used herein “Solvent” or “Solvents” may be any type of those regularly used in chemical synthesis, with the sole limitation that the polymorph Form G and/or Form M of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid may be isolated from the solvent. Among the type of solvents, there may be mentioned low or high boiling point alcohols, ethyl acetate, hexane, isopropyl alcohol, and the like.

The abbreviations used herein are defined according to Aldrichimica Acta, vol. 17, No. 1, 1984. Other abbreviations are defined as follows: “ACN” refers to acetonitrile; “aq” refers to aqueous; “DCM” refers to dichloromethane; “DIEA” refers to diisopropylethylamine; “DSC” refers to differential scanning calorimetry; “DVS” refers to dynamic vapor sorption; “EtOAc” refers to ethyl acetate; “ES/MS” refers to electron spray—mass spectrometry; “h” refers to hour(s); “IPA” refers to isopropanol; “MeOH” refers to methanol; “2-MeTHF” refers to 2-methyltetrahydrofuran; “min” refers to minute(s); “PE” refers to petroleum ether; “prep-HPLC” refers to preparatory high performance liquid chromatography; “RH” refers to relative humidity; “SFC” refers to supercritical fluid chromatography; “TFA” refers to thermogravimetric analysis; “XRPD” refers to X-ray powder diffraction.

### PREPARATION 1

### Preparation of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid

General Preparation of alkylation and hydrolysis: preparation of 2-(methyl(1S,3S)-3-(4-(5,6,7,8-tetrahydro-1,8-naphthyridin-2-yl)butoxy)cylopentyl)amino)-2-((R)-1-methylisochroman-8-yl)acetic acid (Compounds 300 and 299)

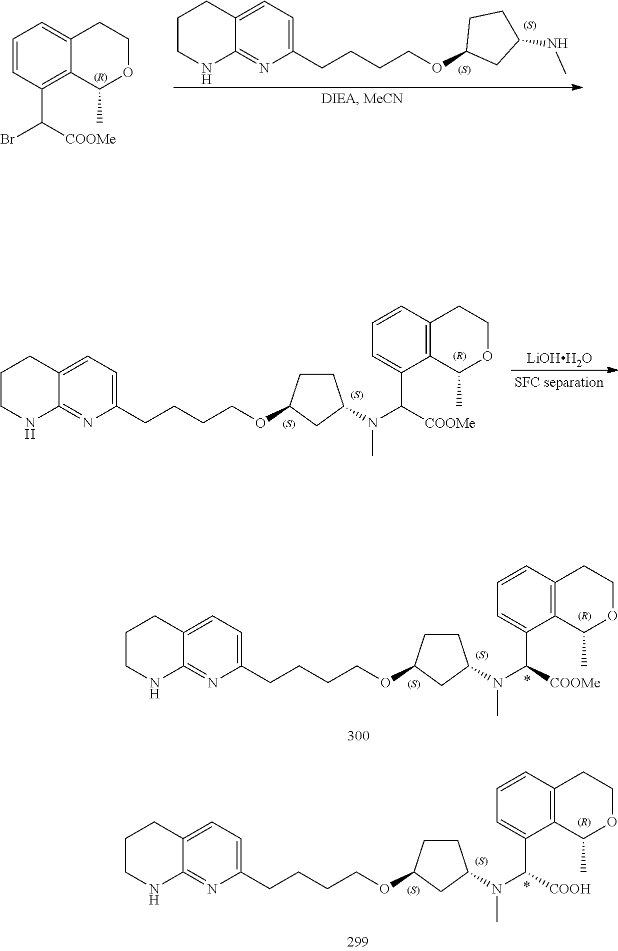

### Step 1 (arbitrary stereochemistry): methyl 2-(methyl((1S,3S)-3-(4-(5,6,7,8-tetrahydro-1,8-naphthyridin-2-yl)butoxy)cyclopentyl)amino)-2-((R)-1-methylisochroman-8-yl)acetate

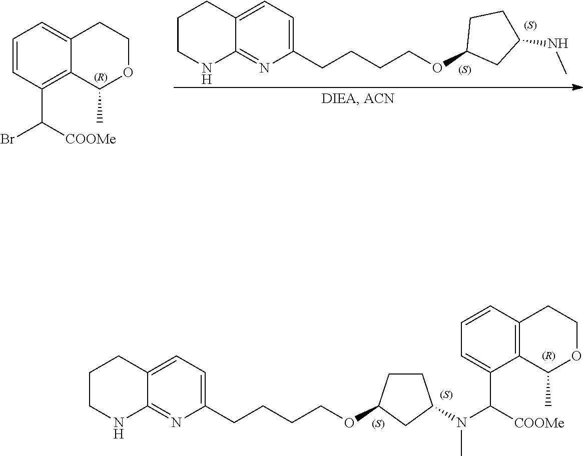

To a solution of (1S,3S)—N-methyl-3-(4-(5,6,7,8-tetrahydro-1,8-naphthyridin-2-yl)butoxy)cyclopentan-1-amine (200 mg, 659.11 μmol, 1 eq) in ACN (4 mL) was added DIEA (255.55 mg, 1.98 mmol, 344.41 μL, 3 eq) and methyl 2-bromo-2-((R)-1-methylisochroman-8-yl)acetate (197.18 mg, 659.11 μmol, 1 eq) at 25° C. under N₂. The mixture was stirred at 80° C. for 12 h. The reaction mixture was quenched by addition H₂O (20 mL) at 20° C., and extracted with EtOAc (10 mL×3). The combined organic layers were washed with brine (20 mL), dried over Na₂SO₄, filtered and concentrated under reduced pressure to give a residue. The residue was purified by column chromatography (SiO₂, PE/EtOAc=I/O to 0/1). Methyl 2-(methyl((1S,3S)-3-(4-(5,6,7,8-tetrahydro-1,8-naphthyridin-2-yl)butoxy)cyclopentyl)amino)-2-((R)-1-methylisochroman-8-yl)acetate (78.52% yield) was obtained as a yellow oil. ES/MS (m/z): 528 (M+1).

### Step 1 (absolute stereochemistry): methyl 2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetate

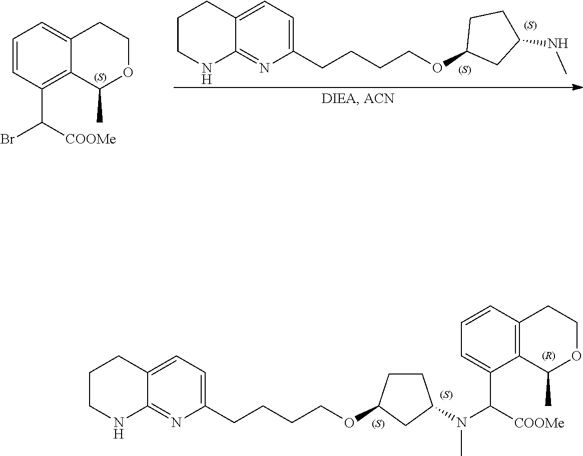

To a solution of (1S,3S)—N-methyl-3-(4-(5,6,7,8-tetrahydro-1,8-naphthyridin-2-yl)butoxy)cyclopentan-1-amine (200 mg, 659.11 μmol, 1 eq) in ACN (4 mL) was added DIEA (255.55 mg, 1.98 mmol, 344.41 μL, 3 eq) and methyl 2-bromo-2-((R)-1-methylisochroman-8-yl)acetate (197.18 mg, 659.11 μmol, 1 eq) at 25° C. under N₂. The mixture was stirred at 80° C. for 12 h. The reaction mixture was quenched by addition H₂O (20 mL) at 20° C., and extracted with EtOAc (10 mL×3). The combined organic layers were washed with brine (20 mL), dried over Na₂SO₄, filtered and concentrated under reduced pressure to give a residue. The residue was purified by column chromatography (SiO₂, PE/EtOAc=1/0 to 0/1). Methyl 2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetate (78.52% yield) was obtained as a yellow oil. ES/MS (m/z): 528 (M+H).

### Step 2 (arbitrary stereochemistry): 2-(methyl((1S,3S)-3-(4-(5,6,7,8-tetrahydro-1,8-naphthyridin-2-yl)butoxy)cyclopentyl)amino)-2-((R)-1-methylisochroman-8-yl)acetic acid (Compounds 300 and 299)

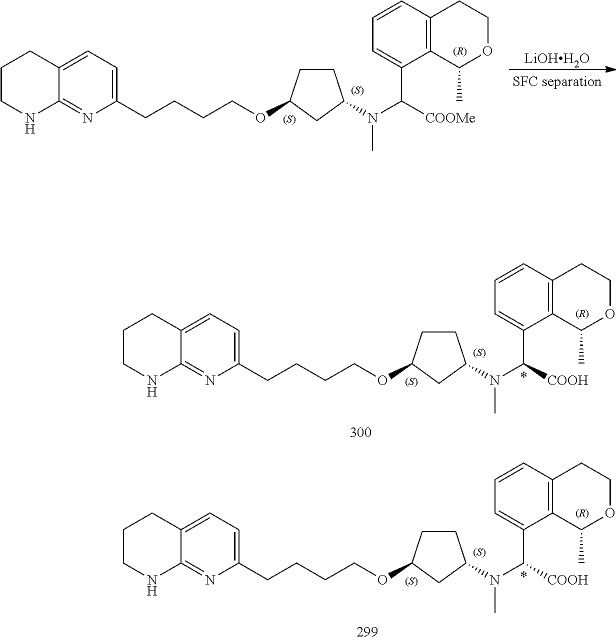

To a solution of methyl 2-(methyl((1S,3S)-3-(4-(5,6,7,8-tetrahydro-1,8-naphthyridin-2-yl)butoxy)cyclopentyl)amino)-2-((R)-1-methylisochroman-8-yl)acetate (250 mg, 479.21 μmol, 1 eq) in DCM (3 mL) in dioxane (4 mL) and H₂O (4 mL) was added LiOH. H₂O (60.32 mg, 1.44 mmol, 3 eq) at 25° C. The mixture was stirred at 35° C. for 12 h. The reaction mixture was concentrated under reduced pressure to give a residue. The residue was purified by prep-HPLC (column: Waters Xbridge Prep OBD C18 150*40 mm*Oum; mobile phase: [NH₄HCO₃-ACN]; gradient: 25%-55% B over 8.0 min). The residue was separated by SFC (column: REGIS (s, s) WHELK-O1 (250 mm*30 mm, 5 um); mobile phase: [CO₂-MeOH (0.1% NH₃H₂O)]; B %:50%, isocratic elution mode) to give two peaks.

### Step 2 (absolute stereochemistry): 2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid (Compounds 300* and 299*)

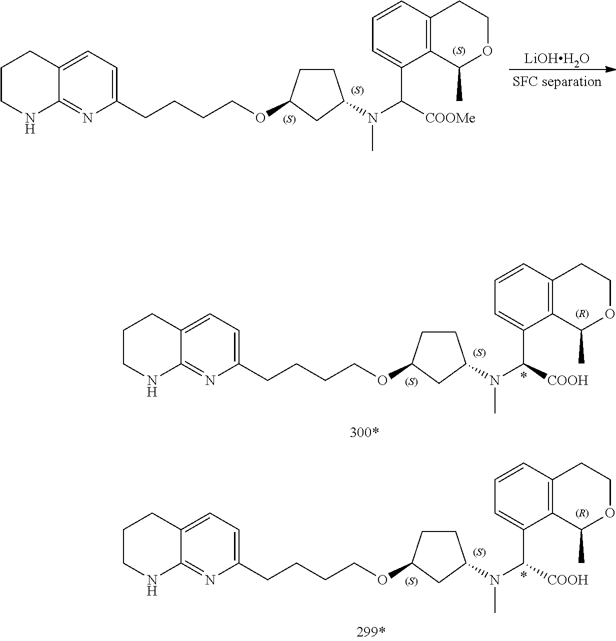

To a solution of methyl 2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetate (250 mg, 479.21 μmol, 1 eq) in DCM (3 mL) in dioxane (4 mL) and H₂O (4 mL) was added LiOH. H₂O (60.32 mg, 1.44 mmol, 3 eq) at 25° C. The mixture was stirred at 35° C. for 12 h. The reaction mixture was concentrated under reduced pressure to give a residue. The residue was purified by prep-HPLC (column: Waters Xbridge Prep OBD C18 150*40 mm*10 um; mobile phase: [NH₄HCO₃-ACN]; gradient: 25%-55% B over 8.0 min). The residue was separated by SFC (column: REGIS (s, s) WHELK-01 (250 mm*30 mm, Sum); mobile phase: [CO₂-MeOH (0.1% NH₃H₂O)]; B %:50%, isocratic elution mode) to give two peaks.

Compound 300 (peak 1), arbitrarily assigned as (S)-2-(methyl((1S,3S)-3-(4-(5,6,7,8-tetrahydro-1,8-naphthyridin-2-yl)butoxy)cyclopentyl)amino)-2-((R)-1-methylisochroman-8-yl)acetic acid, (81.45% yield) was obtained as a white solid. ES/MS (m/z): 508 (M+H). ¹H NMR (400 MHz, CD₃OD) δ=7.62 (d, J=7.4 Hz, 1H), 7.23-7.12 (m, 3H), 6.37 (d, J=7.3 Hz, 1H), 5.27 (q, J=6.5 Hz, 1H), 4.58 (s, 1H), 4.16 (ddd, J=4.6, 9.0, 11.5 Hz, 1H), 3.96 (br s, 1H), 3.89-3.75 (m, 2H), 3.38 (td, J=5.8, 8.9 Hz, 4H), 3.32-3.31 (m, 1H), 2.97-2.88 (m, 1H), 2.84-2.76 (m, 1H), 2.71 (t, J=6.3 Hz, 2H), 2.59-2.49 (m, 2H), 2.47 (s, 3H), 2.10 (br dd, J=7.3, 13.4 Hz, 1H), 2.01-1.90 (m, 2H), 1.90-1.81 (m, 4H), 1.71 (d, J=6.7 Hz, 3H), 1.66-1.58 (m, 2H), 1.56-1.48 (m, 2H).

Compound 299 (peak 2), arbitrarily assigned as (R)-2-(methyl((1S,3S)-3-(4-(5,6,7,8-tetrahydro-1,8-naphthyridin-2-yl)butoxy)cyclopentyl)amino)-2-((R)-1-methylisochroman-8-yl)acetic acid, (13.66% yield) was obtained as a white solid. ES/MS (m/z): 508 (M+H). ¹H NMR (400 MHz, CD₃OD) δ=7.55 (d, J=7.9 Hz, 1H), 7.26-7.19 (m, 2H), 7.12 (br d, J=7.3 Hz, 1H), 6.40 (d, J=7.3 Hz, 1H), 5.59-5.51 (m, 1H), 4.55 (s, 1H), 4.18-4.05 (m, 2H), 3.96 (br s, 1H), 3.84-3.79 (m, 1H), 3.43 (br t, J=6.1 Hz, 2H), 3.38 (br d, J=5.4 Hz, 2H), 2.97-2.90 (m, 1H), 2.83-2.76 (m, 1H), 2.72 (br t, J=6.3 Hz, 2H), 2.68-2.60 (m, 1H), 2.56-2.51 (m, 1H), 2.40 (s, 3H), 2.20-2.12 (m, 1H), 2.09-1.99 (m, 2H), 1.91-1.81 (m, 4H), 1.78-1.64 (m, 4H), 1.60 (d, J=6.6 Hz, 3H), 1.58-1.56 (m, 1H).

When the General Method of Alkylation and Hydrolysis was carried out, the stereochemistry of the starting materials was arbitrarily assigned. Someone skilled in the art will recognize that if the stereochemistry of the starting materials are later determined to be different, the stereochemistry of intermediates and final products may also be determined to be different than those drawn in the scheme. In this example, further analysis led to the reassignment of 299 as (R)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid (Compound 299*), and of 300 as (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid (Compound 300*).

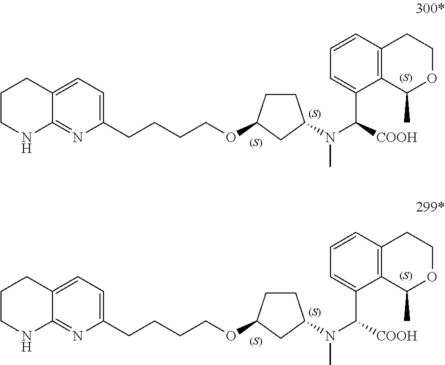

### EXAMPLES

### Example 1

### Naproxen Salt

(S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid HCl solution (6.61 kg corrected free base, 13.0 mol, 1.0 eq) and DCM (80 L, 12 V) were added to the reactor. The pH was adjusted to 8.4-9.5 using 28% NH₄OH. The layers were separated the organic layer was concentrated in vacuo to ~17 L at ≤50° C. EtOAc (99 L, 15 V) was filtered, and the mixture was concentrated in vacuo to ~17 L at ≤50° C. EtOAc (36 L, 5.5 V) was added and the mixture heated to 50° C. The mixture was cooled to 35° C. over ≥30 min and held for ≥30 min. EtOAc (13 L, 2 V) was added, and the mixture was heated to 40° C. The mixture was cooled to 30° C. over ≥30 min. Naproxen (6.91 kg, 2.3 eq) was filtered, and the mixture was cooled to 25° C.≥15 min. (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid naproxen salt was seeded (13.2 g, 0.2 wt %) and the mixture was aged for ≥15 min followed by cooling to 20° C. After aging for 12 h, n-heptane (20 L, 3 V) was added over 2 h and then aged for ≥20 h. The suspension was filtered, and the filter cake was washed with a mixture of EtOAc/n-heptane (3:1, 2×20 L, 2×3 V). The solid was then tray dried in vacuo at 45° C. with a nitrogen sweep. The solid was resuspended in IPA (66 L, 10 V) and was heated to 50° C. over ≥30 min. After aging for ≥2 h the suspension was cooled to 20° C. over ≥2 h. After aging for ≥16 h, the suspension was filtered and the filter cake was washed with IPA (20 L, 3 V). The solid was then tray dried in vacuo at 45° C. with a nitrogen sweep to yield (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl) acetic acid naproxen salt (3.8 kg corrected free base, 57% yield).

### Example 2

### Polymorph Form G

(S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Naproxen salt (3.75 kg corrected free base, 7.37 mol, 1.0 eq) was added to the reactor. 37% HCl (1.63 kg, 2.2 eq) in H₂O (18.8 L, 5 V) was added under nitrogen atmosphere and mixture was heated to 30° C. After 30 min, the mixture was cooled to 20° C. and the aqueous solution was washed with 2-MeTHF (3×16 kg, 3×5 V). DCM (37 L, 10 V) was added to the aqueous solution and the pH was adjusted to 8.4-9.5 with 28% NH₄OH. The layers were separated, and the organic phase was washed with H₂O (19 L, 5 V). The organic phase was concentrated in vacuo to 2.5 V at <50° C. Acetone (57 L, 15 V) was added and the mixture as concentrated in vacuo to 2.5 V at <50° C. Acetone (57 L, 15 V) was added and the mixture as concentrated in vacuo to ~9 L at <50° C. Acetone (3 L, 1 V) was added the mixture was heated to 50° C. After cooling to 45° C., (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Form G seed was added (7.5 g, 0.2 wt %) was added and the mixture stirred for ≥15 min. Acetonitrile (15 L, 4 V) was then added over 1 h while maintaining an internal temperature of 45° C. The mixture was cooled to 20° C. over ≥2 h and then aged for ≥12 h. The suspension was filtered and the filter cake was washed with a mixture of acetone/acetonitrile (1:1, 11 L, 4 V). The solid was then tray dried in vacuo at 65° C. with a nitrogen sweep. Once dry, the solid was hydrated in the tray dryer in the presence of H₂O at 25° C. at atmospheric pressure under nitrogen atmosphere to yield (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Form G (3.18 kg, 85% yield).

### Example 3

### Form G

A prepared sample of the crystalline of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Form G is characterized by an XRPD pattern using CuKα radiation as having diffraction peaks (2-theta values) as described in Table 2 below, and in particular having a peak at 4.6 in combination with one or more of the peaks selected from the group consisting of 11.9 and 17.2; with a tolerance for the diffraction angles of 0.2 degrees.

Confirmation of crystal Form G is shown in Table 1, herein above.

### Example 4

### Crystalline (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid hippurate

### Form A

A prepared sample of the crystalline(S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid hippurate form A is characterized by an XRPD pattern using CuKα radiation as having diffraction peaks (2-theta values) as described in Table 3 below, and in particular having a peak at 10.7 in combination with one or more of the peaks selected from the group consisting of 16.1 and 19.5; with a tolerance for the diffraction angles of 0.2 degrees.

Table 3. X-ray powder diffraction peaks of the XRPD of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Hippurate Form A

TABLE 3

| | (S)-2-(methyl((1S,3S)-3-(4-(5,6,7,8-tetrahydro- | |
|---|---|---|
| | 1,8-naphthyridin-2-yl)butoxy)cyclopentyl)amino)-2- | |
| | ((S)-1-methylisochroman-8-yl)acetic acid Hippurate Form A | |
| | Angle (°2-Theta) | Relative Intensity |
| Peak | +/− 0.2° | (% of most intense peak) |
| 1 | 10.7 | 100.0% |
| 2 | 12.3 | 12.7% |
| 3 | 13.3 | 21.3% |
| 4 | 15.3 | 19.3% |
| 5 | 16.1 | 34.0% |
| 6 | 18.5 | 26.3% |
| 7 | 19.5 | 25.1% |
| 8 | 20.3 | 30.4% |
| 9 | 21.5 | 33.3% |
| 10 | 24.1 | 31.9% |

### Example 5

### Polymorph Form M

To a reactor was added water (41.62 kg, 3.33 V) and 37% aq. HCl (3.60 kg) at 25° C. (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Naproxen salt (12.5 kg, 1.0 eq) was added and the mixture was stirred for 30 min. 2-MeTHF (37.85 kg, 3.9 V) was added and the mixture stirred for 30 min. The layers were separated and the aqueous phase was washed twice with 2-MeTHF (2×37.85 kg, 2×3.9 V). DCM (112 kg, 6.7 V) was added to the aqueous phase and the pH was adjusted to 5.8-6.2 with NH₄OH (1.90 kg, 25% aqueous solution) at 20° C. and stirred for 0.5 h. The phases were separated and the aqueous was back extracted with DCM (54.3 kg, 3.3 V). Water (62.5 kg, 5 V) was added to the combined organics phase and adjusted the pH of aqueous phase to 8.5-8.7 with NH₄OH (0.20 kg, 25% aqueous solution). The layers were separated and the organic phase was washed with H₂O (60 kg, 5 V). The solvent was exchanged to in vacuo to IPA (34 L, 4.5 V). The mixture was warmed to 50° C. to achieve a homogeneous solution. Water (6.4 g, 0.2 eq) was added and stirred for 10 min. (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Form M seed (810 g) was added and stirred for 2 h. Then the mixture was cooled to 40° C. over 3 h and then aged for 49 h. n-Heptane (9.9 kg, 2 V) was added over 2 h at 40° C. and them mixture aged for 5 h. The temperature was cooled to 20° C. over 3 h and the mixture was aged for 28 h. The solid was isolated by filtration and dried in vacuo at 60° C. to yield (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Form M (6.6 kg) in 81% yield.

Form M was stable at 40° C./75% RH and 25° C./96% RH for at least 1 week. The solubility of Form M in simulated fluids (FaSSIF, FeSSIF and FaSSGF) at 37° C. is >4 mg/mL while in water (pH 8.4) at 37° C. is 1.3 mg/mL

### Example 6

### (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form M

A prepared sample of the crystalline Free Form M is characterized by an XRPD pattern using CuKα radiation as having diffraction peaks (2-theta values) as described in Table 2 below, and in particular having a peak at 7.7 in combination with one or more of the peaks selected from the group consisting of 9.7 and 14.8; with a tolerance for the diffraction angles of 0.2 degrees. See, Table 2 herein above.

### Example 7

### Differential Scanning Calorimetry (DSC) for (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Naproxen salt

DSC was conducted using a TA Q2500 DSC run by (and data analyzed by) TA Trios 5.7.0.56. Samples were equilibrated at 25° C. in T-Zero crimped pans, then heated to 300° C. at 10° C./min with a 50 mL/min N₂ purge. The temperature and heat flow were calibrated against indium melting. Enthalpy (normalized): 63.873 J/g. Onset x: 131.91° C. Peak temperature: 135.38° C.

### Example 8

### Thermogravimetric Analysis (TGA) for (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Naproxen salt

TGA was collected using a TA Instruments Q5500 TGA run by (and data analyzed by) TA Trios 5.7.0.56. Samples (~3-10 mg) were heated from ambient temperature (approximately 25° C.) to 200-350° C. at a rate of 10° C./min. The carrier (10 mL/min) and purge (50 mL/min) gas was N₂. The TGA temperature was calibrated by Curie temperature determination with nickel and Alumel standards. The weight calibration was performed with manufacturer-supplied standards. Weight loss: 0.001 mg. Weight percent loss: 0.009%.

### Example 9

### Dynamic Vapor Sorption (DVS) Analysis for (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Naproxen salt

DVS analysis was performed at 25° C. using a TA Instruments Discovery SA flow moisture balance run by (and data analyzed by) TA Trios 5.7.0.56. Sample size was ~5-25 mg, drying 25° C. at 0% RH for 30 minutes, adsorption range 5% to 95% RH, desorption range 95% to 2.5% RH, and step interval 5%. The equilibrium criterion was <0.01% weight change in 5 minutes for a maximum of 30 minutes. Humidity verification calibration was performed with sodium bromide. The weight calibration was performed with manufacturer-supplied standard.

| RH | Weight |
|---|---|
| (%) | Change (%) |
| 5.00 | 0.051 |
| 10.00 | 0.069 |
| 20.00 | 0.102 |
| 80.00 | 0.225 |
| 90.00 | 0.283 |

### Example 10

### ¹³C Solid-State NMR

Solid-state NMR (SSNMR) experiments were performed on a Bruker Avance NEO spectrometer (Bruker, Billerica, MA) equipped with a 9.4 T magnet operating at nominal frequencies of 100.52 MHz for ¹³C and 399.71 MHz for ¹H. A RevNMR HX probe, fitted with a 7 mm magic angle spinning module (Revolution NMR, Fort Collins, CO) was used to acquire the data.

Saturation recovery with ¹³C observation was used to measure ¹H T₁ relaxation times. The ¹H T₁ᵣₕₒ values were determined by ¹³C observation. A pulse delay of 1 second was used between successive acquisitions for the ¹H T₁ measurement for all forms. For the ¹H T₁ᵣₕₒ measurement, pulse delays were optimized based on each form's relaxation properties: 2.5 seconds for polymorph Form G, 7 seconds for polymorph Form M, and 4 seconds for Compound 1 Amorphous

High quality ¹³C spectra were signal averaged for 12 hours using the Cross Polarization Total Sideband Suppression (CPTOSS) sequence with the 243-step phase cycle, and a MAS frequency of 5 kHz with 1.5 ms contact time. One dummy scan prior to acquisition was used, with a ~50 ms of acquisition time. Pulse delays were optimized based on relaxation properties: 2.5 seconds (17,010 acquisitions) for (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form G, 7 seconds (6,318 acquisitions) for (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form M, and 4 seconds (10,692 acquisitions) for (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Amorphous.

3-methylglutaric acid (MGA, KAS-RS-011) was used as a tune-up standard to ensure the spectrometer was operating properly. ¹³C chemical shifts are reported relative to the methyl peak of 3-methylglutaric acid (MGA, KAS-RS-011) at 18.84 ppm with an accuracy of ±0.4 ppm. KAS-RS-011 (3-methylglutaric acid) was purchased from Sigma Aldrich and used without further purification.

The data was processed using Topspin® 4.0.8© software package from Bruker Biospin. The data was Fourier transformed using the full free induction decay (FID) (3960 points) and applying exponential multiplication line broadening (20 Hz for ¹³C CPTOSS spectra, ¹H T₁, and ¹H T₁ᵣₕₒ experiments), phased (apk), and baseline corrected (abs). Manual phasing was performed as needed.

Samples were prepared by packing the compound powders into 7 mm zirconia rotors equipped with PCTFE (Kel-F®) spacers with a nylon screw in the VT hole of the top spacer. The sample material was packed into the NMR rotor under ambient conditions. ³C Solid-state NMR of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph

### Form M

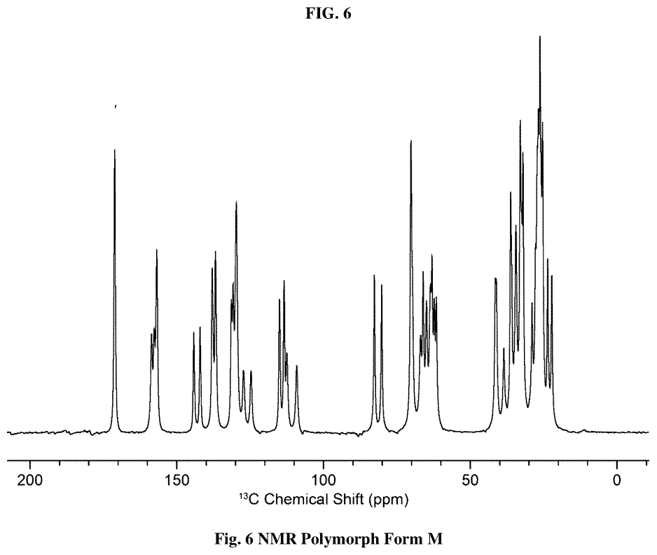

(S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form M is characterized by a solid-state ¹³C NMR spectrum which comprises peaks referenced to the methyl peak of 3-methylglutaric acid (18.8 ppm) at 26.2, 26.7, 27.1, 32.0, 32.9, 25.3, 70.1, and 171.1 ppm, with a tolerance of ±0.4 ppm. An exemplary ¹³C NMR provided as FIG. 6. ¹³C chemical shifts, and normalized intensity relative to the 26.2 ppm ¹³C NMR signal, for (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph Form M—high crystallinity) (Table 6).

TABLE 6
polymorph Form M

| ¹³C Chemical | Normalized Value | NMR |
|---|---|---|
| Shift (ppm) | (d = 26.2 ppm) | Signal Note |
| 28.9 | 32.7 | |
| 32.0 | 70.4 | |
| 32.5 | 56.0 | Shoulder |
| 32.9 | 78.8 | |
| 34.4 | 52.3 | |
| 36.2 | 60.5 | |
| 38.5 | 21.3 | |
| 41.1 | 38.5 | |
| 41.4 | 39.0 | |
| 61.5 | 34.2 | |
| 62.2 | 33.9 | |
| 63.0 | 44.3 | |
| 63.5 | 37.5 | |
| 64.8 | 33.0 | |
| 66.0 | 40.5 | |
| 66.9 | 24.5 | |
| 69.7 | 43.4 | Shoulder |
| 70.1 | 73.7 | |
| 80.1 | 37.2 | |
| 82.6 | 39.8 | |
| 109.1 | 16.7 | |
| 112.4 | 20.0 | |
| 113.4 | 38.3 | |
| 114.9 | 33.8 | |
| 124.6 | 15.5 | |
| 127.2 | 15.6 | |
| 129.6 | 57.8 | |
| 130.8 | 37.4 | |
| 131.3 | 33.5 | |
| 136.8 | 45.6 | |
| 137.9 | 41.6 | |
| 142.0 | 26.6 | |
| 144.1 | 25.5 | |
| 156.8 | 46.1 | |
| 157.6 | 26.4 | |
| 158.5 | 24.9 | |
| 171.1 | 71.2 | |

Note:
Any NMR signal with a normalized intensity less than 3 was not reported.

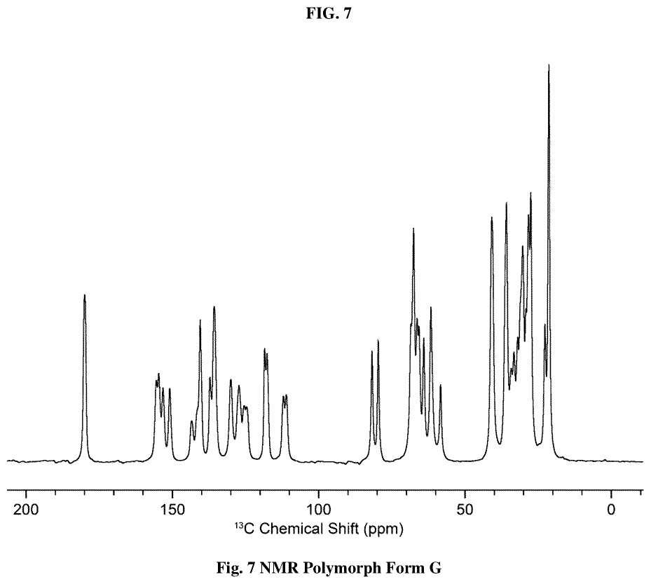

Form G is characterized by a solid-state ¹³C NMR spectrum which comprises peaks referenced to the methyl peak of 3-methylglutaric acid (18.8 ppm) at 21.3, 27.4, 28.2, 35.8, 40.8, 67.5 and 179.9 ppm, with a tolerance of ±0.4 ppm Provided as FIG. 7.b

¹³C chemical shifts, and normalized intensity relative to the 26.2 ppm ¹³C NMR signal, for (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph; Form G (Table 7).

TABLE 7
polymorph Form G

| ¹³C Chemical | Normalized Value | NMR |
|---|---|---|
| Shift (ppm) | (d = 21.3 ppm) | Signal Note |
| 21.3 | 100.0 | |
| 22.6 | 34.7 | |
| 27.4 | 68.0 | |
| 28.2 | 62.2 | |
| 29.1 | 38.6 | |
| 30.2 | 54.4 | |
| 30.9 | 42.3 | Shoulder |
| 31.9 | 31.4 | |
| 33.2 | 27.8 | |
| 34.1 | 23.6 | |
| 35.8 | 65.1 | |
| 40.8 | 61.7 | |
| 58.3 | 19.4 | |
| 61.6 | 39.1 | |
| 64.0 | 31.2 | |
| 65.6 | 34.1 | |
| 66.3 | 36.0 | |
| 67.5 | 58.8 | |
| 68.4 | 34.7 | Shoulder |
| 79.6 | 30.4 | |
| 81.7 | 27.9 | |
| 111.0 | 16.9 | |
| 112.0 | 16.6 | |
| 117.5 | 27.3 | |
| 118.4 | 28.6 | |
| 124.5 | 13.8 | |
| 125.4 | 14.1 | |
| 127.2 | 19.3 | |
| 135.6 | 39.1 | |
| 137.1 | 21.3 | |
| 140.4 | 35.8 | |
| 141.3 | 13.3 | Shoulder |
| 143.3 | 10.3 | |
| 150.8 | 18.5 | |
| 153.1 | 18.6 | |
| 154.6 | 22.3 | |
| 155.4 | 20.4 | |
| 179.9 | 42.1 | |

Note:
Any NMR signal with a normalized intensity less than 3 was not reported.

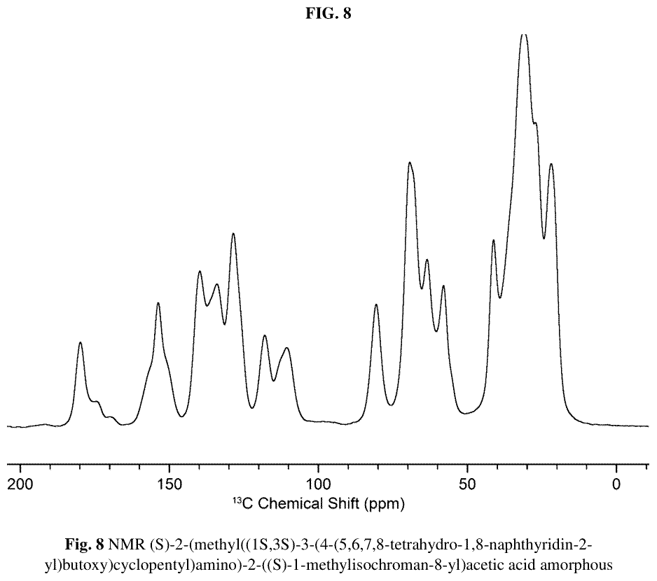

(S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid Amorphous is characterized by a solid-state ¹³C NMR spectrum which comprises peaks referenced to the methyl peak of 3-methylglutaric acid (18.8 ppm) at 21.8, 27.1, 31.0, 58.0, 68.1, 69.3, 153.7, and 179.7 ppm, with a tolerance of ±0.4 ppm, provided as FIG. 8.

We claim:

1. A polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid, or a pharmaceutically acceptable salt; thereof.

2. The polymorph as claimed by claim 1 wherein water is present.

3. The polymorph as claimed by claim 1 wherein a solvent is present.

4. The polymorph as claimed claim 1, characterized by a powder x-ray diffraction pattern using Cu Kα radiation comprising a peak at 4.6°±0.2° 2θ and at least one peak selected from the group consisting of 11.9°, and 17.2°±0.2° 2θ.

5. The polymorph as claimed by claim 4 characterized by a powder x-ray diffraction pattern using Cu Kα radiation comprising peaks at 4.6°, 11.9°, and 17.2°±0.2° 2θ.

6. The polymorph as claimed by claim 5 characterized by a powder x-ray diffraction pattern using Cu Kα radiation comprising peaks at 4.6°, 11.2°, 11.9°, 14.1°, 14.6°, 17.2°, and 22.7°±0.2° 2θ.

7. The polymorph of claim 1, characterized as having an XRPD pattern substantially as shown in FIG. 1.

8. The polymorph as claimed by claim 1, characterized by a solid state ¹³C NMR spectrum comprising a peak at 21.3 ppm, ±0.4 ppm.

9. The polymorph as claimed by claim 8, characterized by a solid state ¹³C NMR spectrum comprising a peak at 21.3 ppm, ±0.4 ppm, and at least one selected from the group consisting of 67.5 and 179.9 ppm±0.4 ppm.

10. The polymorph as claimed by claim 9, characterized by a solid state ¹³C NMR spectrum comprising peaks at 21.367.5, and 179.9 ppm, ±0.4 ppm.

11. The polymorph as claimed by claim 1, characterized as having a ¹³C NMR spectrum substantially as shown in FIG. 7.

12. The polymorph as claimed by claim 1, characterized by a powder x-ray diffraction pattern using Cu Kα radiation comprising a peak at 7.7°±0.2° 2θ, and at least one peak selected from the group consisting of 11.9°, and 17.2°±0.2° 2θ.

13. The polymorph as claimed by claim 12, characterized by a powder x-ray diffraction pattern using Cu Kα radiation comprising peaks at 7.7°, 11.9°, and 17.2°±0.2° 2θ.

14. The polymorph as claimed by claim 13 characterized by a powder x-ray diffraction pattern using Cu Kα radiation comprising peaks at 7.7°, 9.7, 11.9°, 13.5, 14.8, 16.8, and 17.2°±0.2° 2θ.

15. The polymorph as claimed by claim 1, characterized as having an XRPD pattern substantially as shown in FIG. 2.

16. The polymorph as claimed by claim 1, characterized by a solid state ¹³C NMR spectrum comprising a peak at 26.2 ppm, ±0.4 ppm.

17. The polymorph as claimed by claim 16, characterized by a solid state 13C NMR spectrum comprising a peak at 26.2 ppm, ±0.4 ppm, and at least one selected from the group consisting of 70.1 and 171.1 ppm±0.4 ppm.

18. The polymorph as claimed by claim 17, characterized by a solid state 13C NMR spectrum comprising peaks at 26.2, 70.1, and 171.1 ppm, ±0.4 ppm.

19. The polymorph as claimed by claim 1, characterized as having a ¹³C NMR spectrum substantially as shown in FIG. 6.

20. The polymorph as claimed by claim 1, characterized by a solid state ¹³C NMR spectrum comprising a peak at 153.7 ppm, ±0.4 ppm.

21. The polymorph as claimed by claim 20, characterized by a solid state 13C NMR spectrum comprising a peak at 153.7 ppm, ±0.4 ppm, and at least one selected from the group consisting of 31.0 and 58.0 ppm±0.4 ppm.

22. The polymorph as claimed by claim 21, characterized by a solid state 13C NMR spectrum comprising peaks at 31.0, 58.0, and 153.7 ppm, ±0.4 ppm.

23. The polymorph as claimed by claim 1, characterized as having a ¹³C NMR spectrum substantially as shown in FIG. 8.

24. A pharmaceutical composition comprising the polymorph as claimed by claim 1, and a pharmaceutically acceptable carrier.

25. The pharmaceutical composition as claimed by claim 24, wherein the pharmaceutical composition comprises polymorph as claimed by any one of claims 4 to 23 and is substantially free of other polymorphic forms of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid.

26. A method of treating hypertension in a subject in need thereof, comprising administering an effective amount of the polymorph as claimed by claim 1 or the pharmaceutical composition as claimed by claim 24.

27. A method of treating pulmonary arterial hypertension (PAH) in a subject in need thereof comprising administering an effective amount of the polymorph as claimed by claim 1 or the pharmaceutical composition as claimed by claim 24.

28. A method for preparing a polymorph as claimed by claim 1 comprising converting the (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid naproxen salt to a polymorph of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid.

29. A process for the preparation of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid polymorph as claimed by claim 11 wherein there is first prepared a solution of (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid naproxen salt in a solvent or mixture of solvents and thereafter the (S)-2-(methyl((1 S,3S)-3-(4-(5,6,7,8-tetrahydro-1,8-naphthyridin-2-yl)butoxy)cyclopentyl)amino)-2-((S)-1-methylisochroman-8-yl)acetic acid polymorph is precipitated from the solution.

30. A compound that is (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid naproxen salt.

31. A compound that is (S)-2-(methyl((1S,3S)-3-(4-(5,6,7,8-tetrahydro-1,8-naphthyridin-2-yl)butoxy)cyclopentyl)amino)-2-((S)-1-methylisochroman-8-yl)acetic acid hippurate.

32. A compound as claimed by claim 31 characterized by a powder x-ray diffraction pattern using Cu Kα radiation comprising a peak at 10.7°±0.2° 2θ and at least one peak selected from the group consisting of 16.1° and 20.3°±0.2° 2θ.

33. A compound as claimed by claim 32 characterized by powder x-ray diffraction pattern using Cu Kα radiation comprising a peaks at 10.7°, 16.1°, and 20.3°±0.2° 2θ.

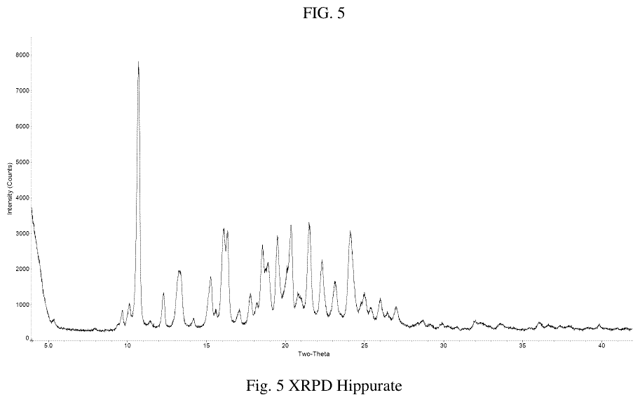

34. A compound as claimed by claim 31 wherein the Hippurate is characterized as having an XRPD pattern substantially as shown in FIG. 5.
